(19) United States
(12) Patent Application Publication
(10) Pub. No.: US 2022/0031158 A1
(43) Pub. Date: Feb. 3, 2022

(54) METHOD FOR DETERMINING AT LEAST ONE GEOMETRICO-MORPHOLOGICAL PARAMETER OF A SUBJECT

(71) Applicant: ESSILOR INTERNATIONAL, CHARENTON-LE-PONT (FR)

(72) Inventors: Gabriel KEITA, DALLAS, TX (US); Arnaud GLACET, DALLAS, TX (US); Benjamin ROUSSEAU, CHARENTON-LE-PONT (FR); Noemi BARRETO, CHARENTON-LE-PONT (FR); Cécile PETIGNAUD, CHARENTON-LE-PONT (FR)

(21) Appl. No.: 17/279,968

(22) PCT Filed: Sep. 24, 2019
(86) PCT No.: PCT/EP2019/075734

§ 371 Date: Mar. 25, 2021

(30) Foreign Application Priority Data
Sep. 26, 2018 (EP) 18306259.5

(51) Int. Cl. A61B 3/11; A61B 3/14; A61B 3/00

(52) U.S. Cl. CPC A61B 3/111; A61B 3/0091; A61B 3/14

(57) ABSTRACT

Disclosed is a method for determining at least one geometrico-morphological parameter of a subject for determining a vision correction equipment, wherein the following steps are performed: a) determining the height of one of the eyes of the subject (P) relative to a reference horizontal surface; b) placing a visual target in front of the head (HP) of the subject (P) at a predetermined position, this predetermined position being determined taking into account the height of one of the eyes of the subject relative to the reference horizontal surface, determined in step a); c) while the subject gazes at the visual target placed at the predetermined position in step b), capturing an image of the head (HP) of the subject (P) with an image capture apparatus (50); and d) deducing from the image captured in step c) the at least one geometrico-morphological parameter.

### TECHNICAL FIELD OF THE INVENTION

The invention relates to a method for determining at least one geometrico-morphological parameter of a subject for a vision correction equipment comprising at least a frame and an ophthalmic lens.

### BACKGROUND INFORMATION AND PRIOR ART

Numerous documents describe devices and methods for measuring geometrico-morphological parameters of a subject such as the interpupillary distance or the fitting heights.

The interpupillary distance is the distance measured between the centers of the pupils of the subject. The fitting heights are the vertical distances between the center of each of the pupil of the subject and the bottom edge of the lens placed in the chosen frame and worn by the subject.

This geometrico-morphological parameters depend on the posture of the subject and on the visual task of the subject. For example, the interpupillary distance of the subject in far vision conditions is different from the interpupillary distance of the subject in near vision conditions.

State of the art for measuring the fitting heights and the interpupillary distance is the manual process done by the eye care professional using a ruler and a pen or a pupilometer.

However, these solutions provide results that seem to demonstrate some variability depending on the eye care professional implementing them.

Alternatively, electronic devices exist. They can be either on a tablet such as the M‘eye’Fit® solution of the applicant, or on a column, such as the VisiOffice® solution of the applicant.

The use of electronic devices removes the manual part of the measurement and globally improves the repeatability of the process. However, some variability may remain due to the remaining manual steps (clip positioning, pupil detection, boxing . . . ), or complex instructions asked to the subject.

### SUMMARY OF THE INVENTION

Therefore one object of the invention is to provide a method for determining at least one geometrico-morphological parameter that exhibits less variable results depending on the eye care professional implementing it.

The above object is achieved according to the invention by providing a method for determining at least one geometrico-morphological parameter of a subject for determining a vision correction equipment comprising at least a frame and an ophthalmic lens, wherein the following steps are performed:

a) determining the height of one of the eyes of the subject relative to a reference horizontal surface,

b) placing a visual in front of the head of the subject at a predetermined position, this predetermined position being determined taking into account said height of one of the eyes of the subject relative to the reference horizontal surface, determined in step a),

c) while the subject gazes at said visual target placed at said predetermined position in step b), capturing an image of the head of the subject with an image capture apparatus,

d) deducing from the image captured in step c) the at least one geometrico-morphological parameter.

Thanks to the method according to the invention, the manual steps that need to be performed and number of instructions that need to be given to the subject are reduced.

Instructions given to the subject are simple and easy to follow.

Consequently, the method generates less error in the measurement. The repeatability is improved.

Other advantageous and non limiting features of the method according to the invention are:
in step a), the height of one of the eyes of the subject is determined for a seating or standing subject;
in step a), the reference horizontal surface is one of the following: the floor, the seat of a chair, or the top surface of a table;
in step a), said height is estimated based on a statistical model linking said height to the total height of the subject;
in step a), said statistical model takes into account the gender of the subject;
in step a), said statistical model takes into account the age of the subject;
in step b), said predetermined position is such that the visual target is positioned approximately at said height of one of the eyes of the subject relative to the reference horizontal surface;
in steps b) and c), said visual target is the entrance pupil of the image capture apparatus;
in steps b) and c), said visual target is different from the entrance pupil of the image capture apparatus and a relative position of said visual target and said entrance pupil of the image capture apparatus is determined;
in step d), a relative position of the visual target and the image capture apparatus is taken into account for determining said geometrico-morphological parameter;
in step d), a relative position of a pupil of one of the eye of the subject and the entrance pupil of the image capture apparatus is taken into account for determining said geometrico-morphological parameter;
in step d), global yaw and pitch angles of the head of the subject while the image is captured are determined and taken into account for determining said geometrico-morphological parameter;
an additional step is performed to determine if both eyes of the subject are looking at the visual target simultaneously, and an alert message is emitted when it is not the case; and
said at least one geometrico-morphological parameter of said subject comprises one of the following: interpupillary distance, half interpupillary distance, fitting height.

The invention also related to a system for determining at least one geometrico-morphological parameter of a subject for determining a vision correction equipment comprising at least a frame and an ophthalmic lens according to the method previously described, comprising:
means for determining the height of one of the eyes of the subject relative to a reference horizontal surface,
a visual target adapted to be placed in front of the head of the subject at a predetermined position, this predetermined position being determined taking into account said height of one of the eyes of the subject relative to the reference horizontal surface, determined by said means,
an image capture apparatus adapted to capture images of the head of the subject, while the subject gazes at said visual target placed at said predetermined position,
calculating means programmed for deducing from this image the at least one geometrico-morphological parameter.

### DETAILED DESCRIPTION OF EXAMPLE(S)

The following description, enriched with joint drawings that should be taken as non limitative examples, will help understand the invention and figure out how it can be realized.

On the appended drawings:

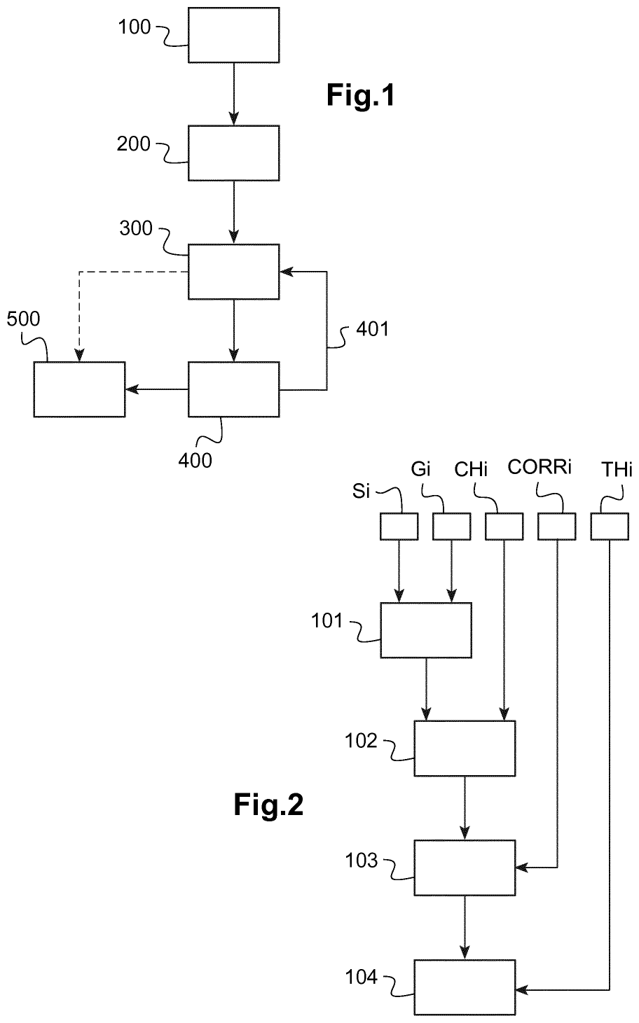

FIG. 1 is a block diagram of the method of the invention,

FIG. 2 is a block diagram of the steps performed in order to determine the position of the image capture apparatus used in the method according to the invention,

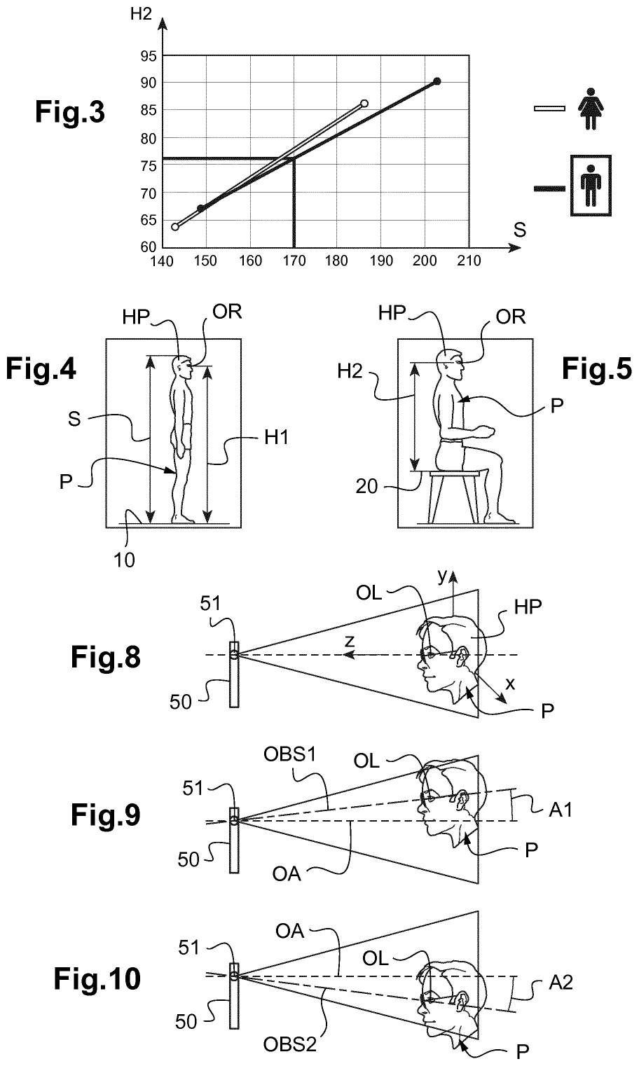

FIG. 3 is a graph showing the eye height when seated as a function of the stature of a subject for male and female subjects,

FIG. 4 is a schematic representation of the total height of the subject,

FIG. 5 is a schematic representation of the eye height when seated of the subject,

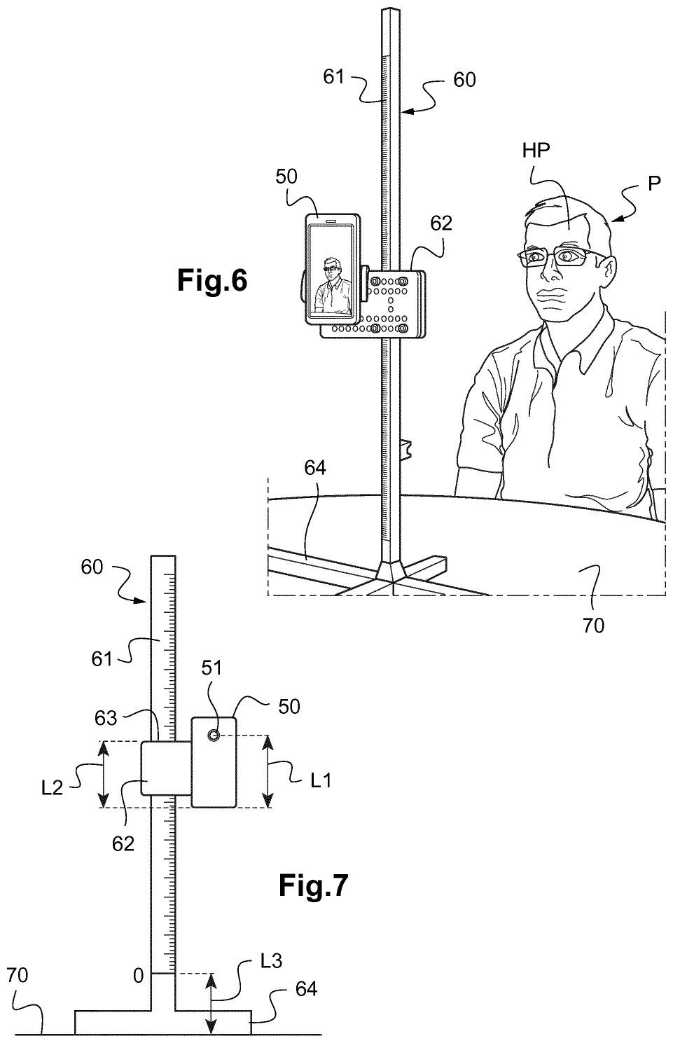

FIG. 6 is a schematic front representation of a measuring device of a system according to the invention,

FIG. 7 is a schematic back representation of the measuring device of FIG. 6,

FIGS. 8 to 10 show different relative positions of the eyes of the subject and the entrance pupil of the image capture apparatus during implementation of the method according to the invention.

In the following, the vertical direction is relative to the direction of a plumb-line. The horizontal direction is orthogonal to the vertical direction. Top and bottom orientation are relative to this vertical direction.

According to the invention, the method for determining at least one geometrico-morphological parameter of a subject for determining a vision correction equipment comprising at least a frame and an ophthalmic lens comprises the following steps:

a) determining the height of one of the eyes of the subject relative to a reference horizontal surface (block 100 of FIG. 1),

b) placing a visual target in front of the head of the subject at a predetermined position, this predetermined position being determined taking into account said height of the eyes of the subject relative to the reference horizontal surface (block 200 of FIG. 1),

c) while the subject gazes at said visual target, capturing an image of the head of the subject with an image capture apparatus (block 300 of FIG. 1),

d) deducing from this image the at least one geometrico-morphological parameter (block 400 of FIG. 1).

Determining a vision correction equipment implies placing ophthalmic lenses adapted to the visual needs of the subject inside a frame chosen by the subject. The ophthalmic lenses must be cut and mounted in the frame in order to ensure that once the frame is placed on the head of the subject, the ophthalmic lens will be adequately placed in front of the eye of the subject in order to provide a accurate correction of the refractive defects of this eye. In particular, the mounting of the lenses in the frame depends on the geometry of the frame, on the morphology of the head of the subject and on the relative position and orientation of the frame and head of the subject.

Thanks to this method, the position of the visual target is determined in a objective manner relying less on the skills of the operator. The variability of the measures achieved based on the image captured while the subject stares at the target is reduced.

Step a)

In step a), the height H1, H2 of at least one of the right and left eyes OR, OL of the subject relative to a reference horizontal surface is determined. The subject may be standing (FIG. 4) or seating (FIG. 5).

The height H1, H2 of the eye OR, OL of the subject is defined as the vertical distance between the pupil of the eye OR of the subject and the reference horizontal surface (FIGS. 4 and 5). Preferably, this distance is determined when the subject looks straight ahead of him in a natural posture. This means that the subject has a primary gaze direction.

The natural posture assumed by the subject is preferably the posture where he looks straight ahead to the horizon without any constraint. This natural posture is also called orthostatic posture and corresponds to the position in which the individual achieves minimal efforts.

In this natural posture, the Frankfurt plane of the head is horizontal or close to be horizontal. For example, the Frankfurt plane forms an angle of 10 degrees or less with a horizontal plane.

The Frankfurt plane is defined as the plane passing through the inferior margin of an orbit (for example, the point called the left orbital) and the upper margin of each ear canal or external auditory meatus, a point called the porion.

In a general manner, the reference horizontal surface is a surface on which a measuring device of a system according to the invention (described in the following) will be standing or is a surface parallel to the surface on which the system will be standing. Preferably, the reference horizontal surface may be any horizontal surface parallel to the seat of the chair whose distance to the seat of the chair is known or may be determined.

In practice, it may be any reference horizontal surface.

If the subject is standing, the reference horizontal surface is for example the floor 10 on which the subject is standing (FIG. 4).

If the subject is seated, the reference horizontal surface is for example the seat 20 of a chair on which the subject is seating (FIG. 5) or an upper surface of a table located close to him, on which a measuring device of a system according to the invention may be placed, as described later.

As it is easy and comfortable for the subject to be seating during the implementation of the method, the case where the subject is seated is of particular interest.

Said height H1, H2 of the eye of the subject relative to the reference horizontal surface is for example estimated on the basis of a statistical model linking said height H1, H2 of the eye relative to the reference horizontal surface to the total height S of the subject (FIG. 4).

The total height of the subject is measured from the top of the head of the subject to the floor. It is his stature.

For example, the US Army Anthropometric Survey II (ANSURII) is among the largest and most comprehensive collection of body size data in the world and enables to create a relation between the total height S of the subject and the height H2 of the eye when seated on a chair relative to the plane of the seat 20 of the chair. These data were collected from 2010 to 2012 and made available to the public in 2017.

In the following, the height H2 of the eye when seated on a chair relative to the plane of the seat 20 of the chair will be named shortly “height of the eye when seated”.

This relation is represented on FIG. 3, showing a graph of the height H2 of the eye when seated as a function of the total height S of the subject.

This study shows that there is a difference between men and women. Advantageously, said statistical model takes into account the gender of the subject.

The ANSUR II study provides a database comprising the height of the eye seated and total height of a population of subjects. The statistical treatment of the data of the ANSUR II study shows that there is a linear relation between the total height and the height of the eye when seated. The statistical model is determined by a linear regression which provides the following formulas for determining the height H2 of the eye when seated as a function of the total height of the subject for men and women:

H2ₘₑₙ=0.3681×S+157.98

H2_{women}=0.3479×S+181.11 (in millimeters).

Similar statistical treatment may for example provide a relation between the height H1 of the eye of the subject when standing, hereafter called the height of the eye when standing, and the total height of the subject.

In an embodiment, said statistical model takes into account the age of the subject.

For example, a correction factor of the height of the eye when seated may be taken into account as a function of the age of the subject. The correction factor is subtracted from the height of the eye when seated determined with the method described previously.

This correction factor is linked to the evolution of the torso posture and spine structure of the subject with age. Studies indeed show that older subjects have lost height because the discs between the vertebrae in the spine dehydrate and compress. The aging spine can also become more curved. Loss of muscle in the torso can also contribute to stooped posture. The correction factor takes into account this evolution to adjust the height of the eyes when seated determined through the statistical model described before.

In practice, the correction factor is for example zero for subjects below 50 years old, 10 mm for subjects 50 to 59 years old, 20 mm for subjects 60 to 69 years old and 30 mm for subjects over 70 years old.

Step b)

In step b), a visual target is placed in front of the head of the subject at a predetermined position, this predetermined position being determined taking into account said height of the eyes of the subject relative to the reference horizontal surface determined in step a).

In order of be accurate, the determination of the geometrico-morphological parameter of the subject is preferably achieved while the subject is in a natural posture, with a primary gaze direction.

In order to facilitate the positioning of the subject into this natural posture, it is useful to place the visual target such that when the subject looks at the visual target, his gaze direction is the primary gaze direction.

To achieve this, said predetermined position is such that the visual target is positioned approximately at said height of the eyes of the subject relative to the reference horizontal surface.

In practice, the predetermined position of the visual target is determined such that it is placed at the same level than the eyes of the subject. In other words, it is placed substantially at the same height than the eyes of the subject relative to a common reference horizontal surface.

Preferably, the entrance pupil of the image capture apparatus is also placed substantially at the same level than the eyes of the subject. If this is not the case (FIGS. 9 and 10), the value of the geometrico-morphological parameters thus determined may be corrected for parallax errors, as described later.

In a particularly advantageous embodiment, the visual target is the entrance pupil of the image capture apparatus.

The image capture apparatus is preferably a portable electronic device such as a smartphone 50 or a computer tablet (FIGS. 6 and 7) with a built in camera.

As described later on, this electronic portable device may be used with a dedicated holder 60 allowing placing the electronic portable device in front of the head HP of the subject P (FIGS. 6 and 7).

In another embodiment, said visual target is different from the entrance pupil of the image capture apparatus and a relative position of said visual target and said entrance pupil of the image capture apparatus is determined.

This relative position may be determined from construction of the set-up used for implementing the method. It can be directly measured.

This step b) will be detailed in a specific embodiment of the method of the invention described later.

Step c)

In step c), while the subject gazes at said visual target, placed in the predetermined position of step b), an image of the head of the subject is captured with the image capture apparatus.

The entrance pupil of the image capture apparatus is placed preferably close to the visual target, if it is different from the visual target.

For example, the visual target is held by the same support that holds the smartphone, just above the top edge of the smartphone.

Preferably, in an additional preliminary step, the subject is instructed to move his head in rotation around a first horizontal axis (vertical rotation movements) and/or around a second vertical axis (horizontal rotation movements), before the image is captured. This is to ensure that the position of the head of the subject is as relaxed as possible.

A dedicated light source or the flash of the portable electronic device may be used to illuminate the head and the eyes of the subject while the image is captured. Illumination and image capture are synchronized.

In particular, the image capture may be synchronized with a flash of light from the flash of the smartphone. Use of the flash improves contrast and image quality. This will therefore ensure an effective automated image processing in step d).

The subject wears the frame chosen for his equipment during the image capture. In order to ensure an accurate determination of the geometrico-morphological parameters, such as the fitting height, in a preliminary step the frame is adjusted to the head of the subject. For example, the position and shape of the temples and/or bridge of the frame are adjusted to center the eyes of the subject relative to the circles of the frame or the ophthalmic lenses.

Preferably, the timer is used to avoid a last movement of the image capture apparatus by the operator, for example, the eye care professional, triggering the image capture. The timer may be set on a three seconds delay, or alternatively on a two seconds delay.

Step d)

In step d), the at least one geometrico-morphological parameter is deduced from the image captured in step d).

Said at least one geometrico-morphological parameter of said subject comprises one of the following: interpupillary distance, half interpupillary distance, fitting height for left and/or right eyes.

The geometrico-morphological parameter is determined based on a treatment of said image captured.

This treatment includes for example improving sharpness and filtering the image in order to improve frame edges visibility.

An additional calibration step may be performed for distortion effect compensation. This additional step may be done during the manufacturing of the portable electronic device used in the measuring device.

It may be also performed before implementing steps c) and d) with a calibrated grid. Several images of the grid are captured at different distances between 40 and 120 centimeters from the grid. Based on these images, it is possible to calculate a compensation matrix for distortion correction. More precisely, considering the pixels of the image that are aligned along two perpendicular directions X and Y, for each pixel of one of these images, a position misalignment of the image of the grid compared to the original grid due to the distortion effect along each of these directions X and Y may be evaluated and used to compensate this distortion effect on the images captured later.

The iris of the eyes may be automatically detected by methods known from the man skilled in the art. Pupils of the subject may also be detected by methods know of the man skilled in the art. If the flash of the portable electronic device is used, reflection of the light of the flash on the cornea may be detected in order to place the center of the pupils.

Detection, on the image captured, of an object of known dimensions, such as a credit card for example, or the frame worn by the subject, or the determination of remarkable points of the face of the subject allows scaling the image.

The interpupillary distance may then be determined based on the distance between the center of the images of the pupils of the eyes and the scale factor of the image.

If iris detection fails, steps c) and d) may be repeated (arrow 401 on FIG. 1).

The boxing system of the frame is defined as the two smallest rectangles that fit best the circles of the chosen frame or the image of the lenses in case of a semi-circled frame or a frame with no circle.

The A size of the frame is the width of this rectangle, corresponding to the horizontal dimension of the circle or lens of the frame when worn by the subject. The B size is the height of the rectangle, corresponding to the vertical dimension of the circle or lens of the frame when worn by the subject.

The A size and B size of the frame may be automatically determined based on the image captured, by measuring the corresponding distances on the image captured and using the appropriate scaling factor.

A size and B size of the frame may then be automatically determined.

If A size and B size of the frame are previously known, the frame may also play the role of the object of known dimensions for scaling the image.

The fitting height may then be determined based on the distance between the center of the image of the pupil and the bottom edge of the image of the frame or of the rectangle corresponding to the frame in the boxing system and the appropriate scaling factor.

If iris detection fails after repeating steps c) and d) a predetermined number of times, the fitting height may be set at a value determined as a function of the size of the frame, for example half of the total height of the circle plus 4 millimeters, that is, half of the length of the side of the rectangle corresponding to the vertical direction of the frame in conditions of wear plus 4 millimeters, or half or the B size of the frame plus 4 millimeters. A message is emitted by the measuring device to require a manual interpupillary distance measurement.

Preferably, in step d),
a relative position of the visual target and the image capture apparatus,
a relative position of the pupil of the eye of the subject and the entrance pupil of the image capture apparatus,
global yaw and pitch angles of the head of the subject while the image was capture,

are determined and taken into account for determining said geometrico-morphological parameter.

More precisely, in the case where the visual target and the entrance pupil of the image capture apparatus are different, the relative position of the head of the subject relative to the image captured is determined. For example, the relative position of at least one of the pupil of the eye of the subject and the entrance pupil of the image capture apparatus may be determined by determining the position of the images of the pupil of the eyes of the subject relative to the edges of the image captured.

If the entrance pupil of the image capture apparatus is at the same height than the eyes of the subject, the image of the pupil of each eye of the subject is vertically centered on the image captured. This means that the images of the pupil of the eye of the subject is centered relative to the top and bottom edges of the image captured, top and bottom corresponding to the orientation of the head on the image.

For example, in the situation of FIG. 8, the images of the head HP and pupils of the subject P are vertically centered in the image captured by the image capture apparatus 50. The entrance pupil 51 of image capture apparatus, here the smartphone 50, is at the same height than the eyes OL of the subject P. The value of the fitting height parameter determined based on this image is accurate.

In the situations of FIGS. 9 and 10, the entrance pupil 51 of the image capture apparatus 50 is not exactly at the same height than the eyes OL of the subject P. The images of the head HP and pupils of the subject P are vertically decentered on the image captured.

In FIG. 9, the entrance pupil 51 of the image capture apparatus 50 is lower than the eyes OL of the subject P. An observation line OBS1 linking the pupil of the eye of the subject to the entrance pupil 51 of the image capture apparatus forms an angle A1 with the optical axis OA of the image capture apparatus 50. Preferably, this angle A1 is equal to or less than 5 degrees.

Without correction, the value of the fitting height parameter determined based on the image captured would be smaller than the accurate value.

In FIG. 10, the entrance pupil 51 of the image capture apparatus 50 is higher than the eyes OL of the subject P. An observation line OBS2 linking the pupil of the eye of the subject to the entrance pupil 51 of the image capture apparatus forms an angle A2 with the optical axis OA of the image capture apparatus 50. Preferably, this angle A2 is equal to or less than 5 degrees.

Without correction, the value of the fitting height parameter determined based on the image captured would be greater than the accurate value.

According to the present invention, the value of the fitting height parameter determined based on images where the images head and eyes of the subject are decentered is corrected by a centering factor. This centering factor may be a linear factor depending on a magnitude representative of the distance between the center of the pupil of the eye OR, OL of the subject P and the entrance pupil 51 of the image capture apparatus 50 in a plane parallel to the image capture plane.

This magnitude may be determined based on the distance, on the image, between the image of the pupil of the subject and a horizontal line equidistant from the top and bottom edge of the image.

Available technologies known from the man skilled in the art allow determining global yaw and pitch angles of the head of the subject while the image was captured, based on the treatment of the image captured.

Yaw angle quantifies the rotation of the head HP of the subject P around a vertical axis y of rotation of the head HP (FIG. 8). Pitch angle quantifies the rotation of the head HP of the subject P around a horizontal axis x of rotation of the head HP (FIG. 8). This horizontal axis of rotation is transverse to the primary gaze direction of the subject. It is for example perpendicular to a sagittal plane of the head.

The value of the pitch angle of the head during an image capture may be used as an indicator of the accuracy of the value of the fitting height parameter determined based on the corresponding image. Alternatively, it may be used to correct the value of the fitting height parameter thus determined.

The value of the yaw angle of the head during an image capture may be used to correct the value of the left and right pupillary distances PDR_{measured}, PDL_{measured} measured between the center of the image pupil of each of the left and right eye to the middle of the image of the bridge of the frame on the corresponding image.

For example, if the value of the yaw angle is different from zero, corrected values PDR_{corrected}, PDL_{corrected} of the right and left pupillary distance may be determined with the following formula:

PDR_{corrected}=PDR_{measured}+CRO·Tan(A_{Yaw})

PDL_{corrected}=PDL_{measured}−CRO·Tan(A_{Yaw})

Where PDR_{measured} and PDL_{measured} are the values of the right and left pupillary distances determined based on the measurement made on the image without correction, CRO is the distance between the center of rotation of the eye and the plane of the frame where the ophthalmic lenses are mounted and A_{yaw} is the value of the yaw angle of the head on said image.

CRO is equal to 25.5 millimeters as an approximation.

An additional step may be performed to determine if both eyes of the subject are looking at the visual target simultaneously, and an alert message is emitted when it is not the case (block 500 of FIG. 1).

Alternatively, in order to determine the geometrico-morphological parameter in conditions of far vision, a correction may be applied to compensate the effect of the convergence of the eyes of the subject on the value of the parameter determined.

During image capture, the subject indeed gazes at a visual target placed about 50 to 80 centimeters away. At this distance DD, the eyes of the subject converge toward the target. The interpupillary distance PD_{measured} determined base on this image is smaller than the interpupillary distance PD_{far} when the subject is in far vision conditions.

The interpupillary distance in far vision conditions may be deduced based on the following formula: PD_{far}=((DD+CRO)/DD)·PD_{measured}. The interpupillary distance is then determined as if it was measured in the plane of the lenses worn by the subject.

As explained before, CRO is equal to 25.5 millimeters as an approximation.

The distance DD between the eye of the subject and the image capture apparatus may be determined based on a calibration of the image capture apparatus, using the known dimensions of the frame worn by the subject, as mentioned before.

Alternatively, the interpupillary distance may be determined as if it was measured in the cornea plane, as it is the case with a pupilometer.

The following formula is then used: PD_{far}=((DD+Rd)/DD)·PD_{measured}, with Rd being equal to the radius of the eye, and approximately equal to 13.5 mm.

Regarding the achievement of step d), an alternative solution is to send the image capture to a remote server having calculations means. The treatment of the image may be achieved with more powerful algorithms. Moreover, the features of the head of the subject and/or of the frame may be compared with a data base of previous measurements stored on this remote server to get a consistent result by comparison.

This remote server is then part of the system used for achieving the method according to the invention.

In the following the implementation of the method described above with a specific dedicated system according to the invention will be described.

Said system for determining at least one geometrico-morphological parameter of a subject for determining a vision correction equipment comprising at least a frame and an ophthalmic lens, comprises:
means for determining the height of one of the eyes of the subject relative to a reference horizontal surface,
a visual target adapted to be placed in front of the head of the subject at a predetermined position, this predetermined position being determined taking into account said height of the eyes of the subject relative to the reference horizontal surface,
an image capture apparatus adapted to capture images of the head of the subject,
calculating means programmed for deducing the at least one geometrico-morphological parameter from said image of the head of the subject captured with said image capture apparatus.

Said means for determining the height of at least one of the eyes of the subject are for example calculating means programmed for determining said height thanks to said statistical model, for example using one of the formulas stated above.

In order to do that, said system also comprises inputting means for inputting the entry data of the statistical model in order to determine said geometrico-morphologico parameter.

These calculation means and inputting means are for example integrated in the portable electronic device used for the image capture. A dedicated application may be for example run on this device. In this example the image capture apparatus is the smartphone 50 already described before.

In the example described here, said visual target is the entrance pupil of the image capture apparatus. This advantageously ensures that both the visual target and the image capture apparatus are place at the most appropriate position relative to the eyes OL, OR of the subject P.

The system according to the invention also comprises a measuring device. Said visual target and image capture apparatus are part of this measuring device. This measuring device is adapted to display said visual target at an adjustable height relative to said reference horizontal surface.

In the example shown in appended FIGS. 6 and 7, said measuring device comprises a holder 60 with a vertical ruler 61 and a support 62 on which said portable electronic device 50 is fixed.

The holder 60 has a foot 64 adapted to be placed on an horizontal surface such as the upper surface of a table to allow the holder 60 to remain standing on said horizontal surface in a stable manner.

Said support 62 is mobile in translation on said ruler 62. It may be blocked by blocking means such as a screw on said ruler 62 at any height on the ruler, from a few centimeters over said horizontal surface on which the holder 60 is placed to a few centimeters below the upper end of the ruler 62.

The embodiment of the corresponding method of the invention comprises the following.

In a preliminary set-up step, the subject is instructed to seat on a chair.

Preferably, this chair is without height adjustment and no arm rest. The geometrical features of this chair, such as the height of the seat of the chair relative to the floor are known. It may be input into the application of the smartphone.

In step a), the height of one of the eyes of the subject relative to the seat of the chair is determined based on the statistical model described before.

The seat of the chair is here the reference horizontal surface.

In step b), the holder 60 with the portable electronic device, for example a smartphone, is placed in front of the head of the subject. The portable electronic device and its support 62 are moved on the ruler 61 to be placed at a predetermined position relative to the ruler 61. This predetermined position is calculated by the application of the smartphone based on the values of entry data.

These entry data are described hereafter.

In practice, the holder 60 is placed on the table 70 that is close to the subject P (FIG. 6). The distance between the eyes of the subject and the entrance pupil of the smartphone 50 fixed on the holder 60, measured in a horizontal plane, is in the range between 40 to 100 centimeters, preferably between 50 to 80 centimeters. As described hereafter, the height of the upper surface of the table 70 is taken into account.

The predetermined position is determined such that the entrance pupil 51 of the back camera of the smartphone 50 is at the height determined in step a). In order to achieve that, the support 62 of the smartphone 50 is translated on the ruler 61 of the holder 60 until a reference edge 63 of the support 60 is placed at a target position on the ruler 61.

More precisely, the steps for the determination of the target position of this reference edge 63 are detailed on FIG. 2 for the example described and represented here.

The determination of the target position of the reference edge or in a general manner of the visual target depends on the specific set-up used.

In a first step of calculation, the total height of the subject and gender of the subject are input in the calculation means (blocks SI, GI of FIG. 2), that is to say, in the application run on said smartphone.

The application is programmed to deduce the height of the eye of the subject seated relative to the seat of the chair, using the appropriate formula given before (block 101 of FIG. 2).

The application is then programmed to take into account the height of the seat of the chair CHI relative to the floor that was input into the application.

It deduces the height of the eye of the subject seated relative to the floor (block 102 of FIG. 2), by adding the height of the seat of the chair to the height of the eye seated relative to the seat of the chair determined.

The application is then programmed to determine a corrected value of this height of the eye of the subject seated relative to the floor taking into account a corrective factor CORRI (block 103 of FIG. 2).

This corrective factor is subtracted to the height of the eye seated relative to the floor. This corrective value allows taking into account the accurate position of the entrance pupil 51 of the back camera of the smartphone 50 used relative to an edge of the smartphone and to the reference edge 63 of the support 62 of the holder 60.

For example, in the situation shown on FIGS. 6 and 7, the corrective factor may be calculated as the distance L2 between the reference edge 63 of the support 60 and the bottom edge of the smartphone minus the distance L1 between the entrance pupil 51 of the back camera of the smartphone 50 and bottom edge of the smartphone 50 minus the distance L3 between the zero mark of the ruler and the upper surface of the table (FIG. 7).

In addition, it may be interesting to add a corrective value of 10 or 20 mm to lower the target position as a function of the age of the subject, as mentioned earlier. In practice, the corrective value is for example zero for subjects below 50 years old, 10 mm for subjects 50 to 59 years old, 20 mm for subjects 60 to 69 years old and 30 mm for subjects over 70 years old.

This corrective value may also be taken into account in the statistical model used.

Finally, the target position of the reference edge 63 of the support 62 of the holder 60 relative to the ruler 61 is determined by subtracting the height THI of the upper surface of the table 70 from the corrected value determined previously (block 104 of FIG. 2).

The operator moves the support 62 along the ruler 61 to place the reference edge 63 of the support 62 on the target position determined.

The subject is instructed to look at the entrance pupil 51 of the image capture apparatus.

He is instructed to move his head in order to avoid unnatural positions of the head.

While the subject gazes at said entrance pupil 51 of the smartphone, at least an image of the head of the subject is captured. The flash is preferably used. The timer is used to avoid any movement of the smartphone when triggering the capture of the image.

Preferably, as shown on FIG. 6, the screen of the smartphone 50 is oriented toward the operator. This allows the operator to roughly check the height of the head of the subject in the image captured by visualizing the image captured on the screen.

Instructions for performing the method may be displayed on said screen when the operator runs the application dedicated to implementing the method according to the invention.

The subject gazes at the entrance pupil 51 of the camera located on the back side of the smartphone 50 (FIG. 7).

In step d), the image of the pupils and/or iris of the subject and the image of the frame worn by the subject are identified in the image captured. The boxing system is determined. The image is scaled. The at least one geometrico-morphological parameter is deduced from the distances measured on the image: the interpupillary distance is deduced from the distance between the image of the center of the pupils or irises of the subject, the fitting height is deduced from the distance between the image of the center of the pupil or iris of the subject and the image of the bottom edge of the frame, the left and right pupillary distances are deduced from the distance between the center of the image of each pupil or iris and the middle of the image of the bridge of the frame.

The geometrico-morphological parameters determined with this embodiment of the method are determined while the subject is looking at a visual target placed 40 to 100 centimeters, preferably 50 to 80 centimeters away, that is to say in conditions of intermediate vision.

Alternatively, the holder 60 may be placed closer of the subject in order to allow determination of the geometrico-morphological parameter in conditions of near vision. The interpupillary distance and right/left pupillary distances, in particular, depend on the visual conditions such as the distance of the visual target. The geometrico-morphological parameter in conditions of far vision may be deduced by a correction of the measured value, as described before. Alternatively, the visual target may be different from the entrance pupil of the image capture apparatus. It may be for example a lighted target such as a diode and it is placed as close as possible from the entrance pupil of the image capture apparatus.

An example of a measuring device of a system of the invention including a target different from an image capture apparatus is a measuring device comprising a measuring column. The measuring column comprises a plurality of diodes aligned on a vertical axis and spaced apart by 1 to 3 centimeters, for example by 2 centimeters. On this vertical axis, the entrance pupil of at least one, preferably at least two image capture apparatuses are placed and regularly space apart. The measuring column stands for example on a table. The subject is for example seated in front of it.

With this measuring column, once the eye height when seated is determined, the diode whose position is closer to this eye height when seated is determined and lighted. It constituted the visual target for the subject. The subject gazes at this lighted diode.

The image capture apparatus closer to the lighted diode is then determined and selected to capture the image of the head of the subject. The image is captured by this image capture apparatus. The distance between the lighted diode and image capture apparatus and therefore the relative position of said visual target and said entrance pupil of the image capture apparatus used is known by construction. This distance is taken into account for determining said geometrico-morphological parameter.

The other steps of the method/parts of the system are unchanged.

Alternatively, a similar method may be implemented with a standing subject.

Alternatively, the height of one of the eyes of the subject relative to a reference horizontal surface and therefore the target position of the smartphone or image capture apparatus may be determined by other methods.

The use of the method and system according to the invention may be associated with the use of an adapted optical design for lenses, having more tolerance to mounting errors. In other words the features of the optical design of a progressive lens such as optical features near the fitting cross and position of the near vision point will be modified in order to ensure a good centering of all the different vision zones when the lens is mounted on a frame using the method according to the invention.

1. Method for determining at least one geometrico-morphological parameter of a subject for determining a vision correction equipment, wherein the following steps are performed:
a) determining the height (H1, H2) of one of the eyes (OL, OR) of the subject (P) relative to a reference horizontal surface,
b) placing a visual target (51) in front of the head (HP) of the subject (P) at a predetermined position, this predetermined position being determined taking into account said height (H1, H2) of one of the eyes (OL, OR) of the subject relative to the reference horizontal surface, determined in step a),
c) while the subject gazes at said visual target (51) placed at said predetermined position in step b), capturing an image of the head (HP) of the subject (P) with an image capture apparatus (50),
d) deducing from the image captured in step c) the at least one geometrico-morphological parameter.

2. The method according to claim 1, wherein, in step a), the height (H1, H2) of one of the eyes (OL, OR) of the subject (P) is determined for a seating or standing subject.

3. The method according to claim 1, wherein, in step a), the reference horizontal surface is one of the following: the floor (10), the seat (20) of a chair, or the top surface of a table.

4. The method according to claim 1, wherein, in step a), said height (H1, H2) is estimated based on a statistical model linking said height to the total height (S) of the subject (P).

5. The method according to claim 4, wherein, in step a), said statistical model takes into account the gender of the subject (P).

6. The method according to claim 4, wherein, in step a), said statistical model takes into account the age of the subject (P).

7. The method according to claim 1, wherein, in step b), said predetermined position is such that the visual target (51) is positioned approximately at said height (H1, H2) of one of the eyes (OL, OR) of the subject relative to the reference horizontal surface.

8. The method according to claim 1, wherein, in steps b) and c), said visual target is the entrance pupil (51) of the image capture apparatus (50).

9. The method according to claim 1, wherein, in steps b) and c), said visual target is different from the entrance pupil of the image capture apparatus and a relative position of said visual target and said entrance pupil of the image capture apparatus is determined.

10. The method according to claim 1, wherein, in step d), a relative position of the visual target and the image capture apparatus is taken into account for determining said geometrico-morphological parameter.

11. The method according to claim 1, wherein, in step d), a relative position of a pupil of one of the eye (OL, OR) of the subject (P) and the entrance pupil (51) of the image capture apparatus (50) is taken into account for determining said geometrico-morphological parameter.

12. The method according to claim 1, wherein, in step d), global yaw and pitch angles of the head (HP) of the subject (P) while the image is captured are determined and taken into account for determining said geometrico-morphological parameter.

13. The method according to claim 1, wherein an additional step is performed to determine if both eyes (OL, OR) of the subject (P) are looking at the visual target simultaneously, and an alert message is emitted when it is not the case both eyes of the subject are not looking at the visual target simultaneously.

14. Method according to claim 1, wherein said at least one geometrico-morphological parameter of said subject comprises one of the following: interpupillary distance, half interpupillary distance, fitting height.

15. System for determining at least one geometrico-morphological parameter of a subject for determining a vision correction equipment comprising at least a frame and an ophthalmic lens according to the method of claim 1, comprising:
means for determining the height of one of the eyes of the subject relative to a reference horizontal surface,
a visual target adapted to be placed in front of the head (HP) of the subject (P) at a predetermined position, this predetermined position being determined taking into account said height (H1, H2) of one of the eyes (OL, OR) of the subject (P) relative to the reference horizontal surface, determined by said means,
an image capture apparatus (50) adapted to capture images of the head of the subject, while the subject gazes at said visual target placed at said predetermined position,
calculating means programmed for deducing from this image the at least one geometrico-morphological parameter.

16. The method according to claim 2, wherein, in step a), the reference horizontal surface is one of the following: the floor (10), the seat (20) of a chair, or the top surface of a table.

17. The method according to claim 5, wherein, in step a), said statistical model takes into account the age of the subject (P).

18. The method according to claim 2, wherein, in step b), said predetermined position is such that the visual target (51) is positioned approximately at said height (H1, H2) of one of the eyes (OL, OR) of the subject relative to the reference horizontal surface.

19. The method according to claim 3, wherein, in step b), said predetermined position is such that the visual target (51) is positioned approximately at said height (H1, H2) of one of the eyes (OL, OR) of the subject relative to the reference horizontal surface.

20. The method according to claim 4, wherein, in step b), said predetermined position is such that the visual target (51) is positioned approximately at said height (H1, H2) of one of the eyes (OL, OR) of the subject relative to the reference horizontal surface.
